(19) United States
(12) Patent Application Publication
(10) Pub. No.: US 2005/0220895 A1
(43) Pub. Date: Oct. 6, 2005

(54) Use of gallium to treat inflammatory arthritis

(76) Inventors: Louis R. Bucalo, Miami Beach, FL (US); Sunil Sreedharan, San Francisco, CA (US); Krishna Allamneni, Sunnyvale, CA (US); Lawrence R. Bernstein, Menlo Park, CA (US)

Correspondence Address: REED INTELLECTUAL PROPERTY LAW GROUP, 1400 PAGE MILL ROAD, PALO ALTO, CA, 94304-1124, US

(21) Appl. No.: 11/015,172

(22) Filed: Dec. 17, 2004

Related U.S. Application Data
(60) Provisional application No. 60/530,353, filed on Dec. 17, 2003.

(51) Int. Cl. A61K 31/33; A61K 31/555; A61K 33/24

(52) U.S. Cl. 424/617; 514/184; 514/492

(57) ABSTRACT

Methods are provided for the use of gallium in the treatment or prevention of inflammatory arthritis conditions such as rheumatoid arthritis.

### CROSS REFERENCE To RELATED APPLICATIONS

This application claims priority under 35 U.S.C. § 119(e)(1) to U.S. Provisional Application Ser. No. 60/530,353, filed Dec. 17, 2003, the disclosure of which is incorporated by reference in its entirety.

### TECHNICAL FIELD

The present invention relates generally to the treatment or prevention of inflammatory arthritis.

### BACKGROUND

Arthritis literally means inflammation of a joint, and can cause pain, stiffness and sometimes swelling in or around joints. Major types of arthritis include osteoarthritis, caused by wear and tear, and inflammatory arthritis, which consists of several disease conditions, ranging from relatively mild forms such as ‘tennis elbow’ and bursitis to crippling systemic forms, such as rheumatoid arthritis. Common types of inflammatory arthritis include rheumatoid arthritis, ankylosing spondylitis, systemic lupus erythematosus, psoriatic arthritis, and juvenile rheumatoid arthritis.

The common denominator of all these rheumatic diseases is autoimmune related joint and musculoskeletal pain and related systemic effects. The abnormal immune response is responsible for the inflammation of the tissues lining the joint, breakdown of the joint cartilage, and the loosening of the ligaments and tendons supporting the joint. In addition, ongoing inflammation also causes the synovial membrane to grow into a thick, abnormal, invading tissue referred to as a pannus. All of these processes result in destruction of the cartilage, underlying bone surrounding the joint, ligaments, and tendons, and formation of abnormal bone due to periosteal proliferation to compensate for the bone loss, eventually leading to deformed joints.

Because these autoimmune diseases are systemic in nature, other tissues and organs are also affected. For example, inflamed or enlarged nerves, lymph nodes, sclera, pericardium, spleen, arteries and rheumatoid nodules are frequent components of the disease. In addition, the potential exists for involvement of the kidney, lung, and the cardiovascular systems. Ankylosing spondylitis is a chronic inflammation of the spine and the sacroiliac joint (the point where the spine meets the pelvic bone) that can also cause inflammation in other joints. Systemic lupus erythematosus, or lupus, is an autoimmune disease in which the body harms its own healthy cells and tissues. Juvenile rheumatoid arthritis is a form of arthritis similar to rheumatoid arthritis that affects young children, and results in inflamed, swollen joints that can be stiff and painful. The cause of this disease is also considered to be autoimmune in nature but is otherwise poorly understood. However, unlike adults with rheumatoid arthritis, most children with juvenile rheumatoid arthritis do not have long-term disease and disability, and go on to lead healthy adult lives. Juvenile rheumatoid arthritis is often referred to as juvenile idiopathic arthritis, due to its unknown cause.

Rheumatoid arthritis is an autoimmune disease; the trigger for the disease is not known, but a genetic factor may increase the risk of developing rheumatoid arthritis. It is a systemic disease typically affecting multiple joints on both sides of the body simultaneously, and the synovial membrane lining the joints. The symptoms of rheumatoid arthritis include pain, stiffness, and swelling in the joints of the hands, wrists, elbows, feet, ankles, knees, and/or neck. This inflammation may destroy the joint tissues over time. Therefore, physicians typically recommend early treatment with medication to either control the disease or prevent its progression, since worsening of the condition can lead to permanent disability.

Gallium maltolate and related gallium hydroxypyrones are described in U.S. Pat. No. 5,258,376 to Bernstein. These are orally bioavailable gallium compounds with broad clinical potential in a variety of diseases including cancer (U.S. Pat. No. 6,087,354 to Bernstein), bone disease (U.S. Pat. No. 5,998,397 to Bernstein) and infectious disease. Steady-state serum levels of gallium, as well as favorable bioavailability in animal models in patients have been safely achieved, thus establishing that orally administered gallium is bioavailable without instigating adverse systemic toxicity.

Gallium has shown anti-inflammatory and immunomodulating activity in some in vitro and animal models of autoimmune disease, inflammatory disease, and allograft rejection. The data suggest that clinical testing of gallium may be warranted for treating inflammatory arthritis, and in particular, but not limited to, the treatment of autoimmune-based arthritis such as rheumatoid arthritis, psoriatic arthritis, and lupus. Bernstein (1998) Pharmacol. Rev. 50:665-682.

U.S. Pat. No. 5,175,006 to Matkovic et al. describes the use of gallium compounds, and gallium nitrate, in particular, for the treatment of arthritis. Gallium nitrate was administered subcutaneously in the rheumatoid arthritis rat adjuvant model. It was determined that administration of 0.5-4 mg of gallium nitrate per kg of body weight was necessary to achieve a therapeutic steady state concentration in blood. However, the steady state concentrations achieved are not specified. See also Matkovic et al. (1991) Curr. Ther. Res. 50:255-267.

There are numerous commercial products available for the treatment of inflammatory arthritis. However, there remains a need for the development of improved therapies. For example, most rheumatoid arthritis therapies include multiple drugs prescribed based on the extent and severity of the disease. Patients with early stages of rheumatoid arthritis are started on milder non-steroidal anti-inflammatory drugs or Cox-2 inhibitors and, as the disease progresses, other more potent and potentially more toxic drugs, such as steroids or disease-modifying anti-rheumatic drugs, are layered in.

Due to serious side effects, it is highly desirable to reduce patient reliance on both steroids and conventional disease-modifying antirheumatic drugs such as the cytotoxic agent, methotrexate. In addition, newer biologics are replete with limitations such as drug or metabolite related systemic toxicity, weight loss, reduced efficacy with long-term usage, allergic drug reactions, liver failure, glucose intolerance, high cost, lack of insurance coverage etc. Most of these therapies do not cure the disease and have significant potential side effects or other shortfalls. In addition, many known therapeutics take weeks, and even months, to show measurable therapeutic benefits.

Fortunately, there are recent animal models for arthritis and rheumatoid arthritis, which have been useful in identifying “potential” therapeutic agents. See Bendele et al. (1999) Toxicologic Pathology 27(1):134-142 and Bendele (2001) J. Musculoskel. Neuron. Interact. 1(4):377-385. However, animal models typically only provide data as to a compounds' activity and toxicity, and many compounds that exhibit a capacity for disease modification often can result in unacceptable toxicity during prolonged dosing in the clinical setting.

Therefore, there remains a need for the development of therapeutics to treat inflammatory arthritis that do not have the problems associated with current therapies, and which are not toxic during prolonged dosing. These needs are addressed by the methods of the invention, where the effect of gallium at the serum levels attained was observed relatively quickly, i.e. within days.

### SUMMARY OF THE INVENTION

One aspect of the invention relates to a method of treating inflammatory arthritis and rheumatic diseases comprising administering to a patient in need thereof, a therapeutically effective amount of gallium, wherein the therapeutically effective amount provides a gallium blood serum level within the range of approximately 50-7000 ng/ml.

Another aspect of the invention relates to a methods of preventing pannus formation, preventing periosteal proliferation, preventing cartilage damage, splenomegaly, hepatomegaly, and preventing bone resorption due to inflammatory arthritis, comprising administering a therapeutically effective amount of gallium to a patient in need thereof.

### BRIEF DESCRIPTION OF THE DRAWINGS

FIGS. 1-8 provide data obtained from the adjuvant-induced acute arthritis model of Example 1.

FIGS. 1 and 2 show the effect of oral gallium, delivered as gallium maltolate, on ankle inflammation, with FIG. 1 showing the gross pathology of the ankle upon clinical observation, and FIG. 2 showing the histological scores of ankle inflammation. Higher scores reflect more severe degrees of swelling and inflammation.

FIG. 3 shows the effect of oral gallium delivered as gallium maltolate on paw weight as a reflection of joint inflammation and edema.

FIG. 4 shows the histological score of bone damage, with higher scores reflecting more severe bone resorption.

FIG. 5 shows the effect of oral gallium delivered as gallium maltolate on body weight. Arthritic animals lose body weight due to loss of mobility that impacts feeding. Dexamethasone negatively impacted this body weight loss induced by the ankle swelling while gallium had a favorable effect, although both reduced ankle inflammation.

FIGS. 6 and 7 show the effect of oral gallium delivered as gallium maltolate on liver and spleen weight, respectively. A gallium dose-related decrease in the arthritis-induced liver and spleen weights can be observed.

FIG. 8 shows the spleen histopathology score. Gallium significantly reduced inflammation in the spleen and prevented atrophy of the lymphoid tissue that develops during the course of the disease.

FIGS. 9-14 provide data obtained from the streptococcal cell wall-induced chronic arthritis model of Example 1.

FIGS. 9 and 10 show the effect of oral gallium delivered as gallium maltolate on ankle inflammation by clinical and histological evaluations, respectively. With the first reactivation, gallium (300 mg/kg) reduced the swelling significantly on day 12. The second reactivation (flare-up) on day 14 resulted in the swelling peaking 2 days later. Gallium treated rats had decreased ankle swelling starting within 2 days of the flare-up, with the peak effect observable by 6 days after the flare-up. Cyclosporine had no effect. Histologically, there was a dose-related inhibition of inflammation scores.

FIG. 11 shows the dose-related effect of oral gallium delivered as gallium maltolate on periosteal proliferation (abnormal formation of new bone).

FIG. 12 shows the dose-related effect of oral gallium delivered as gallium maltolate on pannus (abnormal proliferation of synovial tissue).

FIG. 13 shows the effect of oral gallium delivered as gallium maltolate on cartilage damage.

FIG. 14 shows the effect of oral gallium delivered as gallium maltolate on abnormal bone resorption (destruction of bone).

### DETAILED DESCRIPTION OF THE INVENTION

Prior to discussing the invention in further detail, the following terms will be defined. Unless defined below, the terms used herein have their normally accepted meanings.

The term “administering” refers to the administration of any conventional form for the delivery of a pharmaceutical composition to a patient that results in the gallium being present in the blood stream. The portion of the administered dose that is absorbed in the blood stream is referred to as the “bioavailable fraction” and can readily be determined by techniques known in the art, such as, for example, by measuring the blood serum level.

The term “therapeutically effective” amount of a drug means a sufficient, nontoxic amount of a compound to provide the desired effect at a reasonable benefit/risk ratio. The desired effect may be alleviation of the signs, symptoms, or causes of a disease, or any other desired alteration of a biological system. In particular, a therapeutically effective amount refers to an amount of gallium complex administered such that a blood serum gallium concentration is obtained that is sufficient to enable treatment or prevention of the disease state of interest. The therapeutically effective amount necessary to prevent a disease is referred to as the “prophylactically effective amount.”

The term “therapeutic agent” refers to any additional therapeutic agent that is co-administered with gallium in the methods of the invention. The additional therapeutic agent can be administered by any route or in any dosage form. Co-administration can be by simultaneous, overlapping, or sequential administration. Simultaneous administration can be in the form of separate or combined dosage forms. In one preferred embodiment, the combined dosage form is suited for oral administration.

The term “treat,” as in “to treat a condition,” includes (1) preventing the condition, i.e., avoiding any clinical symptoms of the condition, (2) inhibiting the condition, that is, arresting the development or progression of clinical symptoms, and/or (3) relieving the condition, i.e., causing regression of clinical symptoms.

The term “patient”, as in “treatment of a patient”, is intended to refer to an individual human or other mammal afflicted with or prone to a condition, disorder, or disease as specified herein.

The term “pharmaceutically acceptable” means a material that is not biologically or otherwise undesirable, i.e., the material may be administered to an individual along with the gallium (and any additional therapeutic agents) without causing any undesirable biological effects or interacting in a deleterious manner with any of the other components of the pharmaceutical composition in which it is contained.

“Optional” or “optionally” means that the subsequently described circumstance may or may not occur, so that the description includes instances where the circumstance occurs and instances where it does not. For example, recitation of an additive as “optionally present” in a formulation herein encompasses both the formulation containing the additive and the formulation not containing the additive.

It must be noted that as used herein and in the claims, the singular forms “a”, “and”, and “the” include reference to both the singular and plural unless the context clearly dictates otherwise. Thus, for example, reference to “a therapeutic agent” in a formulation includes two or more active agents, reference to “a carrier” includes two or more carriers, and so forth.

### Pharmaceutical Compositions and Modes of Administration

The methods of this invention are achieved by using a pharmaceutical composition comprising gallium. Suitable forms of gallium include, gallium acetate, gallium carbonate, gallium citrate, gallium chloride, gallium fluoride, gallium formate, gallium nitrate, gallium oxylate, gallium oxide and hydrated gallium oxide, gallium phosphate, gallium tartrate, gallium-pyridoxal isonicotinoyl hydrazone, tris(8-quinolinolato)gallium (III), neutral 3:1 gallium complexes of a 3-hydroxy-4-pyrone, gallium (III) complexes of an N-heterocycle, and gallium salt complexes of polyether acids.

In one embodiment of the invention, the gallium is a neutral 3:1 gallium complex of a 3-hydroxy-4-pyrone. The term “neutral 3:1 gallium complex of a 3-hydroxy-4-pyrone” refers to an electrostatically neutral complex of Ga³⁺ (Ga(III)) and three equivalents of the anionic form of a 3-hydroxy-4-pyrone, which complex is represented by the formula [Ga³⁺ (py⁻)₃], wherein py⁻ represents the anionic form of a 3-hydroxy-4-pyrone as defined below. Because such complexes do not dissociate to any significant extent in aqueous solutions maintained at a pH of from about 5 to about 9, these complexes remain predominantly electrostatically neutral in such solutions.

The term “3-hydroxy-4-pyrone” refers to a compound of Formula I: 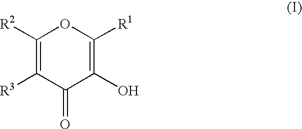
wherein R¹, R², and R³ are independently selected from H and —C₁₋₆ alkyl. The —C₁₋₆ alkyl group can be branched or unbranched but is preferably unbranched. Suitable —C₁₋₆ alkyl groups include, by way of illustration and not limitation, methyl, ethyl, isopropyl, and n-propyl. Preferred —C₁₋₆ alkyl groups are those having 1-3 carbons, in particular, methyl, and ethyl. Single substitution is preferred, particularly substitution at the 2- or the 6-position, with substitution at the 2-position being most preferred. Exemplary compounds encompassed by the term “a 3-hydroxy-4-pyrone” are described as follows. The unsubstituted form of Formula I (R¹, R², and R³ are H) is known as pyromeconic acid. Compounds of Formula I where R² and R³ are H include: 3-hydroxy-2-methyl-4-pyrone (R¹ is —CH₃), which is also known as maltol or larixinic acid; and 3-hydroxy-2-ethyl-4-pyrone (R¹ is —C₂H₅), which is sometimes referred to as ethyl maltol or ethylpyromeconic acid. Both of these are preferred for use in the methods of the invention, in particular 3-hydroxy-2-methyl-4-pyrone. Compounds of Formula I where R¹ and R³ are H include 3-hydroxy-6-methyl-4-pyrone (R² is —CH₃). The term “an anion of a 3-hydroxy-4-pyrone” refers to a compound defined in Formula I above wherein the hydroxyl proton has been removed so as to provide for the anionically charged form of the compound. These neutral 3:1 gallium complexes and their method of synthesis are described in U.S. Pat. No. 6,004,951 to Bernstein.

Preferred complexes include, by way of illustration and not limitation, the 3:1 complex of maltol with gallium, which is referred to as tris(3-hydroxy-2-methyl-4H-pyran-4-onato) gallium or gallium maltolate; and the 3:1 complex of ethyl maltol with gallium, referred to as tris(3-hydroxy-2-ethyl-4H-pyran-4-onato)gallium or gallium ethyl maltolate.

In another embodiment of the invention, the gallium is a gallium (III) complex of an N-heterocycle, having Formula (II) 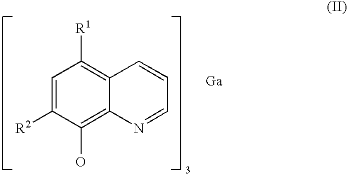
wherein R¹ is selected from hydrogen, halo, and —SO₃M where M is a metal ion, and R² is selected from hydrogen, or R¹ is chloro and R² is iodo. Exemplary metal ions include potassium and sodium. These gallium (III) complexes of N-heterocycles and their method of synthesis are described in U.S. Pat. No. 5,525,598 to Collery et al.

In another embodiment of the invention, the gallium is a gallium salt complex of a polyether acid, for example gallium 3,6-dioxaheptanoate. These salts can be synthesized in a manner similar to that set forth in U.S. Pat. Nos. 6,054,600 and 6,303,804, both to Dougherty et al. One example of a suitable gallium salt complex of a polyether acid is a compound of formula (III): 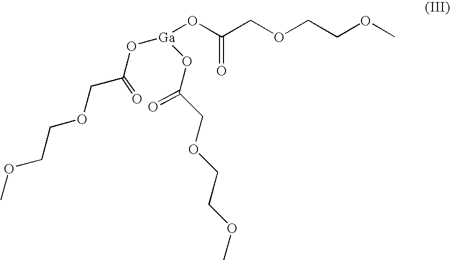
Typically, the polyether acid will have the formula: CH₃O(CH₂CH₂O)ₙCH₂COOH, where n is an integer from 0 to 2. The gallium complex can be prepared by reaction of a gallium alkoxide and a polyether acid anhydride, where the anhydride is prepared from its corresponding polyether acid. Exemplary gallium alkoxides have the formula GA(OR)₃, where R is a substituted and unsubstituted straight or branched C₁₋₈alkyl or aryl group. Exemplary anhydrides of polyether acids include 3,6-dioxaheptanoic acid anhydride.

In another embodiment of the invention, the gallium is tris(8-quinolinolato)gallium (III), which is described in Theil et al. (1999) Relevance of tumor models for anticancer drug development, Contrib. Oncol. (Feibig and Burger, eds, Basel, Karger) and in Coller et al. (1996) Anticancer Res. 16:687-692. Gallium pyridoxal isonicotinoyl hydrazone is also of interest, and is described in Knorr et al. (1998) Anticancer Res. 18:1733-1738 and Chitambar et al. (1996) Clin Can Res 2:1009-1015.

The compounds may be administered orally, parenterally (including by subcutaneous, intravenous, and intramuscular injection), transdermally, rectally, nasally, opthalmically, buccally, sublingually, topically, vaginally, etc., in dosage formulations typically containing one or more conventional pharmaceutically acceptable carriers. In one preferred embodiment, the route of administration is oral and the gallium is an orally bioavailable form of gallium such as, by way of example and not limitation, a neutral 3:1 gallium complex of a 3-hydroxy-4-pyrone or a gallium (III) complex of an N-heterocycle.

Depending on the intended mode of administration, the pharmaceutical compositions may be in the form of solid, semi-solid, or liquid dosage forms, such as, for example, tablets, suppositories, pills, capsules, powders, liquids, suspensions, creams, ointments, lotions, or the like, preferably in unit dosage form suitable for single administration of a precise dosage. The compositions contain an effective amount of gallium, generally although not necessarily in combination with a pharmaceutically acceptable carrier and, in addition, may include other pharmaceutical agents, adjuvants, diluents, buffers, etc.

The actual dosage may vary depending upon the gallium compound administered and the dosage can be selected so as to provide a predetermined amount of Ga(III) to be delivered per kilogram of patient weight. For example, the methods of the invention may involve administering a gallium compound that provides about 0.1 to 20 mg Ga(III)/kg, preferably about 1 to 20 mg Ga(III)/kg, and more preferably about 1 to 12 mg Ga(III)/kg.

As noted above, preferred compositions herein are oral formulations, which include delayed release oral formulations. For oral dosage forms, while gallium is delivered to the bloodstream from the gastrointestinal tract, partial dissociation may occur under acidic conditions (generally at a pH of about 4 or less). Such acidic conditions may be present in the stomach. The dissociation may result in formation of less absorbable complexes, together with free hydroxypyrone and ionic gallium. Accordingly, in order to maintain an orally delivered gallium in a form that is highly absorbable in the gastrointestinal tract, the pharmaceutical compositions of this invention may be formulated to contain a means to inhibit dissociation of this complex when exposed to the acidic conditions of the stomach. Means to inhibit or prevent dissociation of this complex when exposed to the acidic conditions of the stomach are described, for example, in U.S. Pat. No. 6,004,951 to Bernstein. Suitable compositions can include a buffering agent, while another means of inhibiting or preventing dissociation is to encapsulate the pharmaceutical composition in a material that does not dissolve until the small intestine of the individual is reached, such as with enteric coated tablets, granules, or capsules, as is well known in the art.

### Methods of Pharmaceutical Treatment

As noted above, the present invention is directed to methods for treating and preventing inflammatory arthritis and rheumatic diseases by administering gallium. Examples of types of inflammatory arthritis to which the methods of the invention find utility include, by way of illustration and not limitation, rheumatoid arthritis, ankylosing spondylitis, and systemic lupus erythematosus.

The method of the invention finds particular utility in the treatment of primary and secondary inflammatory arthritis, which includes by way of illustration and not limitation, rheumatoid arthritis, ankylosing spondylitis, psoriatic arthritis, juvenile rheumatoid arthritis, Reiter's Syndrome and enteropathic arthritis. In addition, the methods of the invention are useful in treating other rheumatic diseases, including but not limited to, systemic lupus erythematosus, systemic sclerosis and scleroderma, polymyositis, dermatomyositis, temporal arteritis, vasculitis, polyarteritis, Wegener's Granulomatosis and mixed connective tissue disease. Prophylactic treatment is also contemplated for these disease states.

Thus, one embodiment of the invention relates to treating inflammatory arthritis and rheumatic diseases by administering to a patient in need thereof, a therapeutically effective amount of gallium. The therapeutically effective amount provides a gallium blood serum level within the range of approximately 50-7000 ng/ml. See, for example, FIGS. 1, 2, 9, and 10, where gallium is shown to reduce ankle inflammation.

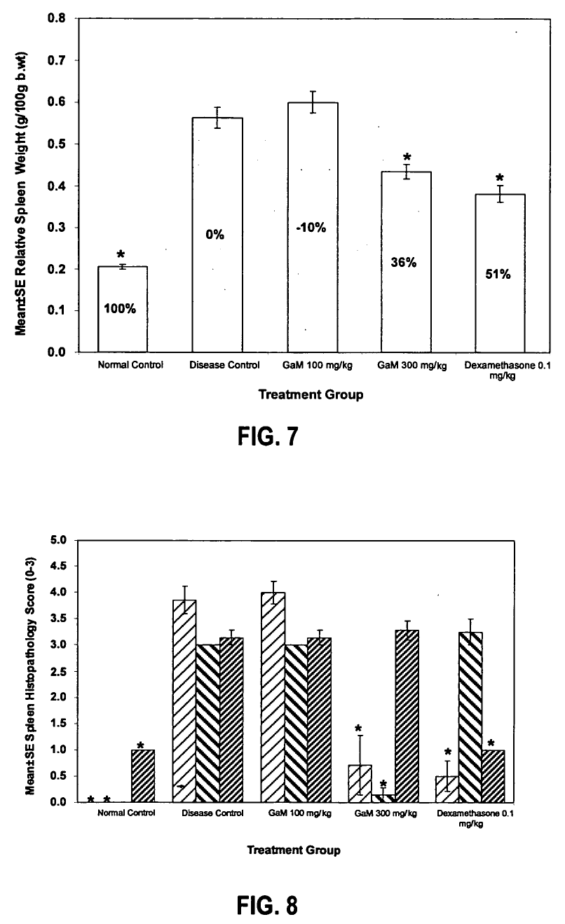

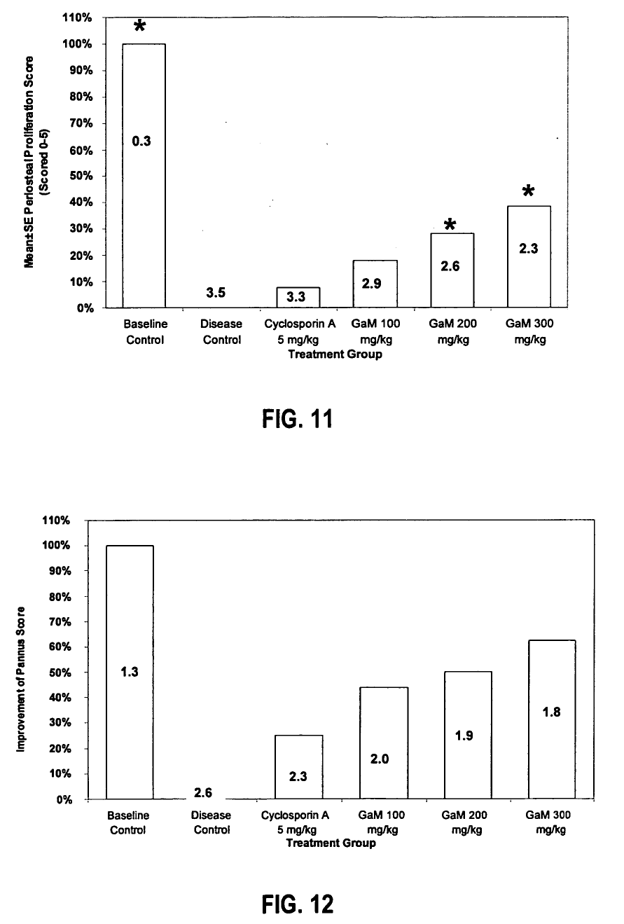

There are numerous pathological conditions associated with inflammatory arthritis. Evaluation of a chronic arthritis model has shown that gallium has beneficial effects on: periosteal proliferation, which is the abnormal formation of new bone (FIG. 11); pannus, which is the abnormal proliferation of synovial tissue that subsequently invades the underlying cartilage and bone (FIG. 12); cartilage damage (FIG. 12); splenomegaly, which is enlargement of the spleen (FIGS. 7 and 8); hepatomegaly, which is enlargement of the liver due to the hypertrophy or increase in the size of liver cells (FIG. 6); and abnormal bone resorption, which is the destruction of bone (FIG. 14). Accordingly, the methods of the invention are also directed to the use of gallium in the prevention of pannus formation, periosteal proliferation, cartilage damage, splenomegaly, hepatomegaly, and to prevent bone resorption.

In one embodiment of the invention, the methods provide a therapeutic effect of gallium within about 60 days, preferably within about 30 days, more preferably within about 14 days, and most preferably within about 7 days after administration.

The gallium is preferably administered in single dose form, but may be administered in multiple doses per day. The gallium is preferably administered at least one hour before meals and at least two hours after meals, but other schedules are also acceptable.

Optionally, it may be desired to include additional active agents with the gallium. Such additional agents include, by way of example and not limitation, non-steroidal anti-inflammatory drugs such as but not limited to acetaminophen, aspirin, diclofenac, fenoprofen, flurbiprofen, ibuprofen, indomethacin, ketoprofen, meclofenamate, nabumetone, naproxen, oxaprozin, piroxicam, sulindac, tolmetin, celecoxib, rofecoxib and valdecoxib; glucocorticoids such as but not limited to cortisone, dexamethasone, prednisolone, prednisone, or triamcinolone; immunosuppressive drugs such as but not limited to azathioprine, cyclophosphamide, cyclorporine and methotrexate; disease modifying antirheumatic drug therapies such as but not limited to gold compounds, hydroxychloroquine, leflunomide, penicillamine or sulfasalazine; and biological agents such as but not limited to the anti-tumor necrosis factor agents and interleukin-1 receptor antagonists, adalimumab, anikinra, etanercept, infliximab and mabthera; and combinations thereof.

It is to be understood that while the invention has been described in conjunction with the preferred specific embodiments thereof, the foregoing description, as well as the examples that follow, are intended to illustrate and not limit the scope of the invention. Other aspects, advantages, and modifications will be apparent to those skilled in the art to which the invention pertains.

All patents, patent documents, and publications cited herein are hereby incorporated by reference in their entirety for their disclosure concerning any pertinent information not explicitly included herein.

### EXAMPLES

The following examples are put forth so as to provide those of ordinary skill in the art with a complete disclosure and description of how to make and use the compounds of this invention, and are not intended to limit the scope of what the inventor regards as his invention.

Efforts have been made to ensure accuracy with respect to numbers (e.g., amounts, temperature, etc.) but some errors and deviations should be accounted for. Unless otherwise indicated, parts are parts by weight, temperature is in degrees Celsius, and pressure is at or near atmospheric. All solvents were purchased as HPLC or reagent grade and, where appropriate, solvents and reagents were analyzed for purity using common techniques.

### Example 1

Two preclinical animal models were tested for oral gallium efficacy in inflammatory polyarthritis; adjuvant-induced acute arthritis and streptococcal cell wall-induced chronic arthritis, respectively. Male Lewis rats were used in both studies. The models are described in detail in Bendele et al. (1999) Toxicologic Pathology 27(1):134-142 and Bendele (2001) J. Musculoskel. Neuron. Interact. 1(4):377-385.

### Adjuvant-Induced Acute Arthritis Model

Materials and methods: For the adjuvant-induced acute arthritis model, male Lewis rats (7 per group for Gallium Maltolate, 4 per group for normal controls and dexamethasone-treated controls) were injected with 100 μl of Freund's complete adjuvant/lipoidal amine (FCA/LA) subcutaneously at the base of the tail on study day 0 under anesthesia. The rapid onset (within 7 days) of arthritic symptoms in this model includes ankle inflammation, bone resorption, and mild cartilage destruction. Prophylactic treatment was initiated by dosing with control vehicle or Gallium Maltolate (100 or 300 mg/kg) by daily oral gavage, from seven days prior to adjuvant injection until termination. The dexamethasone-treated control animals were administered a daily oral dose of dexamethasone (0.1 mg/kg). Body weights were measured regularly during the course of the study to track the effect of the drugs on the weight loss induced by the developing adjuvant disease, and dose volumes were adjusted accordingly. Prior to the onset of swelling, but after the establishment of systemic disease (about 7 days after adjuvant injection), caliper measurements were made of ankle joints. Ankles were measured every day until 14 days post-adjuvant injection when the rats were anesthetized and euthanized. Serum was harvested one hour after final dosing for gallium quantitation. Hind paws, liver and spleen were weighed, fixed and processed for histopathologic evaluation. Adjuvant arthritic ankles were given scores of 0-5 (0=normal; 5=severe) for inflammation and bone resorption. Splenic changes of inflammation, increased extramedullary hematopoiesis and lymphoid atrophy were scored 0-5 using criteria similar to those used for scoring of inflammation. The primary endpoint is periarticular inflammation and bone resorption as quantitated by ankle caliper measurements and histopathologic evaluation of ankles (scoring of joints). Secondary endpoints include body weight change and the inhibition of splenomegaly and hepatomegaly.

Results: Following daily oral gavage of 100 or 300 mg/kg of oral gallium delivered as gallium maltolate in suspension with 1% methyl cellulose, the results indicated that: repeated administration for 14 days in Lewis rats was safe and showed no signs of toxicity; serum gallium levels attained were dose-dependent; a significant reduction in clinical and histological ankle inflammation, bone resorption scores at both doses; and a marked reduction in liver and spleen hypertrophy at both doses indicates the onset of relief from symptoms.

The data is shown in FIGS. 1-8. FIG. 1 shows ankle diameter of rats with adjuvant-induced acute arthritis treated with gallium maltolate (GaM), dexamethasone, or vehicle (normal and disease controls). Results expressed as the mean ankle diameter±standard error (SE) for treatment groups. Results are also expressed numerically as the percent difference from the disease control group, n=4 rats for normal control and dexamethasone treated groups, n=7 for other treatment groups, *p<0.05 compared with disease control group.

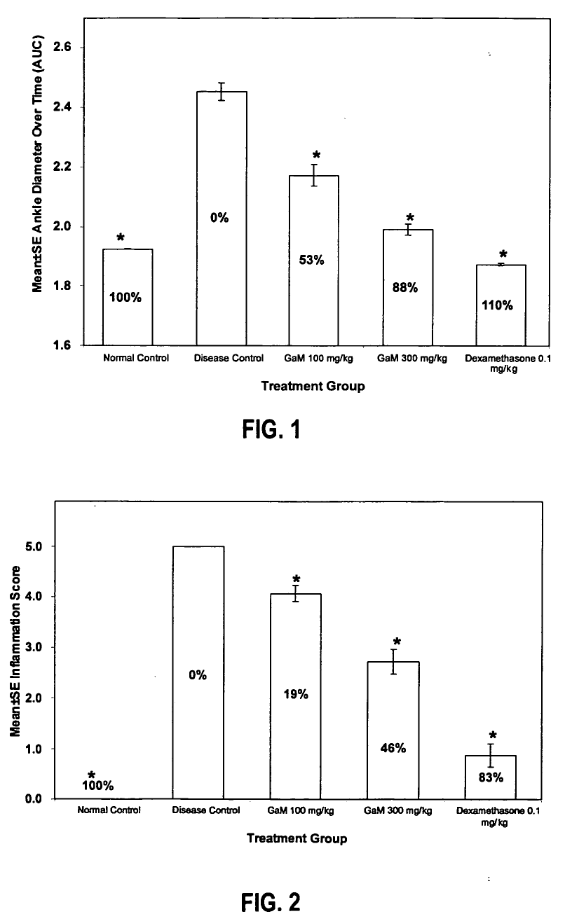

FIG. 2 shows the inflammation scores for rats with adjuvant-induced acute arthritis treated with gallium maltolate (GaM), dexamethasone, or vehicle (normal and disease controls). Results are expressed as the mean score±SE. Score scale: normal=0, minimal change≦1, mild change≦2, moderate change≦3, marked change≦4, and severe change=5. Results are also expressed numerically as the percent difference from the disease control group, n=4 rats for normal control and dexamethasone treated groups, n=7 for other treatment groups, *p<0.05 compared with disease control group.

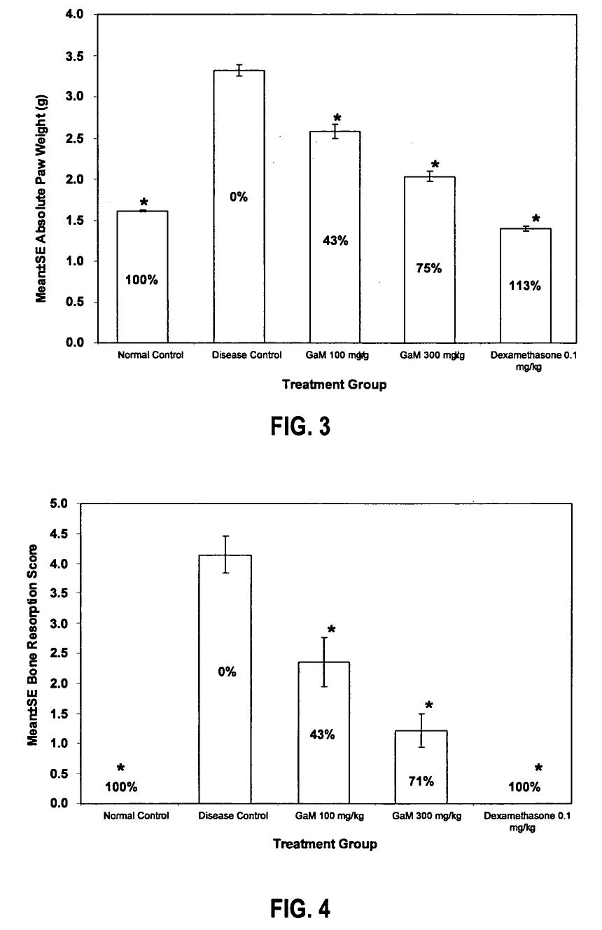

FIG. 3 shows paw weight of rats with adjuvant-induced acute arthritis treated with gallium maltolate (GaM), dexamethasone, or vehicle (normal and disease controls). Results are expressed as the mean paw weight (g)±standard error (SE) for treatment groups. Results are also expressed numerically as the percent difference from the disease control group, n=4 rats for normal control and dexamethasone treated groups, n=7 for other treatment groups, *p<0.05 compared with disease control group.

FIG. 4 shows bone resorption scores of rats with adjuvant-induced acute arthritis treated with gallium maltolate (GaM), dexamethasone, or vehicle (normal and disease controls). Results are expressed as the mean score

$${\pm SE .}^{•}$$

Score scale: normal=0, minimal change≦1, mild change≦2, moderate change≦3, marked change≦4, and severe change=5. Results are also expressed numerically as the percent difference from the disease control group, n=4 rats for normal control and dexamethasone treated groups, n=7 for other treatment groups, *p<0.05 compared with disease control group.

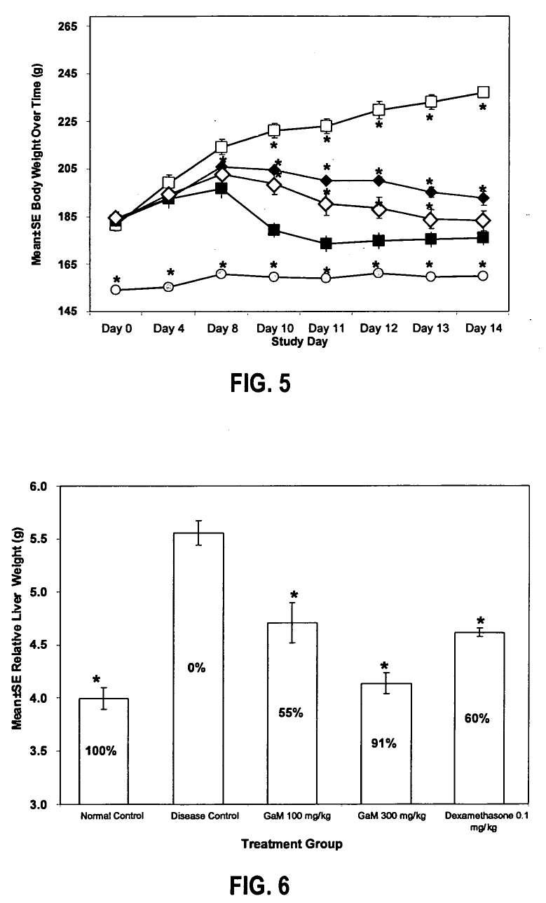

FIG. 5 shows body weight of rats with adjuvant-induced acute arthritis treated with gallium maltolate (GaM), dexamethasone, or vehicle (normal and disease controls). Results are expressed as the mean body weight (g)±standard error (SE) for treatment groups at various times in the study. Results are also expressed numerically as the percent difference from the disease control group, n=4 rats for normal control and dexamethasone treated groups, n=7 for other treatment groups, *p<0.05 compared with disease control group.

FIG. 6 shows liver weight of rats with adjuvant-induced acute arthritis treated with gallium maltolate (GaM), dexamethasone, or vehicle (normal and disease controls). Results are expressed as the mean liver weight (g)±standard error (SE) for treatment groups. Results are also expressed numerically as the percent difference from the disease control group, n=4 rats for normal control and dexamethasone treated groups, n=7 for other treatment groups, *p<0.05 compared with disease control group.

FIG. 7 shows spleen weight of rats with adjuvant-induced acute arthritis treated with gallium maltolate (GaM), dexamethasone, or vehicle (normal and disease controls). Results are expressed as the mean relative spleen weight (g/100 g of body weight)±standard error (SE) for treatment groups. Results are also expressed numerically as the percent difference from the disease control group, n=4 rats for normal control and dexamethasone treated groups, n=7 for other treatment groups, *p<0.05 compared with disease control group.

FIG. 8 shows spleen histopathology scores of rats with adjuvant-induced acute arthritis treated with gallium maltolate (GaM), dexamethasone, or vehicle (normal and disease controls). Results are expressed as the mean score for inflammation, lymphoid atrophy or extramedullary hematopoiesis

$${\pm SE .}^{•}$$

Score scale: normal=0, minimal change≦1, mild change≦2, moderate change≦3, marked change≦4, and severe change=5, n=4 rats for normal control and dexamethasone treated groups, n=7 for other treatment groups, *p<0.05 compared with disease control group.

In summary, in the acute model for adjuvant-induced arthritis, oral gallium delivered as gallium maltolate was safe with no signs of toxicity observed after 14 days of daily administration. Significant dose dependent protection from adjuvant induced joint inflammation was observed.

### Streptococcal Cell Wall-Induced Chronic Arthritis Model

This is a multiple reactivated peptidoglycan-polysaccharide (PGPS)-induced arthritis model. The rapid onset (4-5 days) of arthritic symptoms in this model includes ankle inflammation, bone resorption, mild cartilage destruction.

Materials and methods: Male Lewis rats (N=12/group) with developing streptococcal (PGPS) cell wall induced arthritis were treated with gallium maltolate (100, 200 or 300 mg/kg, po, qd) or Cyclosporin A (CSA, 5-20 mg/kg) prophylactically beginning 1 day after intra-articular injection of PGPS into the ankles (day −14) and continued for 14 days at which time systemic reactivation was induced by intravenous (iv) injection of PGPS (day 0). Treatment was continued for another 14 days and animals were reactivated a second time (day 14). Following an additional week of treatment, rats were terminated for a total of 34 days of dosing. Rats were weighed on days (−)13, (−)7, 0, 8, 14 and 21, at which time, dose volumes were adjusted. Right ankle caliper measurements were taken on days 0, 2, 4, 6, 8, 10, 12, 14, 16, 18, 20 and 21. Since arthritis was observed in left hind paws on day 18, additional caliper measurements were take for left ankles on days 18, 20 and 21. All rats had terminal blood samples obtained for PK sampling. Scoring of Joints: PGPS arthritic ankles are given scores of 0-5 (normal to severe) for inflammation, pannus, cartilage damage, bone resorption and periosteal bone proliferation according criteria similar to the acute arthritis study. The primary endpoint is periarticular inflammation and bone resorption as quantitated by ankle caliper measurements and histopathologic evaluation of ankles (scoring of joints).

Results: Following daily oral gavage, beginning on day −13, of 100, 200 or 300 mg/kg of oral gallium delivered as gallium maltolate in suspension with 1% methyl cellulose, the results indicated that: repeated administration of 100, 200, and 300 mg/kg oral gallium delivered as gallium maltolate for 35 days in Lewis rats was safe and showed no signs of toxicity; after the first reactivation of arthritis on day 0, a slight inhibition of inflammation was detected in animals treated with 300 mg/kg oral gallium; after the second reactivation, all oral gallium treated groups had decreased paw weights and ankle swelling. The effects were most significant at higher oral gallium doses; and joint histopathology showed dose responsive inhibition (20-45%) of the sum of the scores for inflammation, pannus, cartilage damage and bone damage, indicating the onset of relief from symptoms.

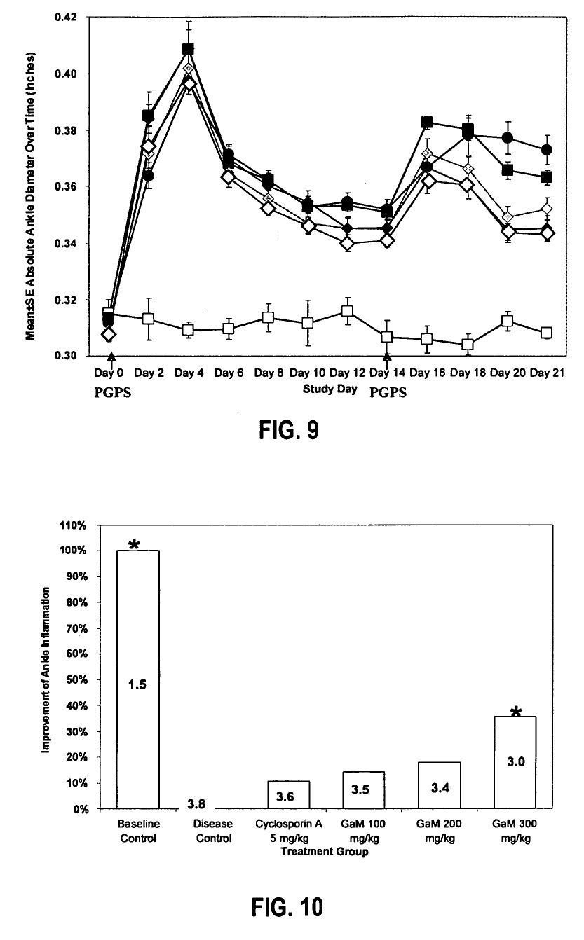

The data is shown in FIGS. 9-14. FIG. 9 shows ankle diameter of rats with PGPS-induced chronic arthritis treated with gallium maltolate (GaM), cyclosporine A, or vehicle (baseline or disease controls). Results are expressed as the mean ankle diameter (inches)±standard error (SE) at various times in the study. Arrows indicate PGPS induction, n=4 rats for baseline control group, n=12 rats for disease control and treatment groups.

FIG. 10 shows percent improvement of ankle inflammation in rats with PGPS-induced chronic arthritis treated with gallium maltolate (GaM), cyclosporin A, or vehicle (normal and disease controls). Results are expressed as the mean percent difference from disease controls

$${\pm SE .}^{•}$$

Results are also expressed numerically as score of ankle inflammation on the scale: normal=0, minimal change≦1, mild change≦2, moderate change≦3, marked change≦4, and severe change=5, n=4 rats for baseline control group, n=12 rats for disease control and treatment groups, *p<0.05 compared with disease control group.

FIG. 11 shows percent improvement of periosteal proliferation in rats with PGPS-induced chronic arthritis treated with gallium maltolate (GaM), cyclosporin A, or vehicle (normal and disease controls). Results are expressed as the mean percent difference from disease controls

$${\pm SE .}^{•}$$

Results are also expressed numerically as score of periosteal proliferation on the scale: normal=0, minimal change≦1, mild change≦2, moderate change≦3, marked change≦4, and severe change=5, n=4 rats for baseline control group, n=12 rats for disease control and treatment groups, *p<0.05 compared with disease control group.

FIG. 12 shows percent improvement of pannus proliferation in rats with PGPS-induced chronic arthritis treated with gallium maltolate (GaM), cyclosporin A, or vehicle (normal and disease controls). Results expressed as the mean percent difference from disease controls

$${\pm SE .}^{•}$$

Results are also expressed numerically as score of pannus proliferation on the scale: normal=0, minimal change≦1, mild change≦2, moderate change≦3, marked change≦4, and severe change=5, n=4 rats for baseline control group, n=12 rats for disease control and treatment groups.

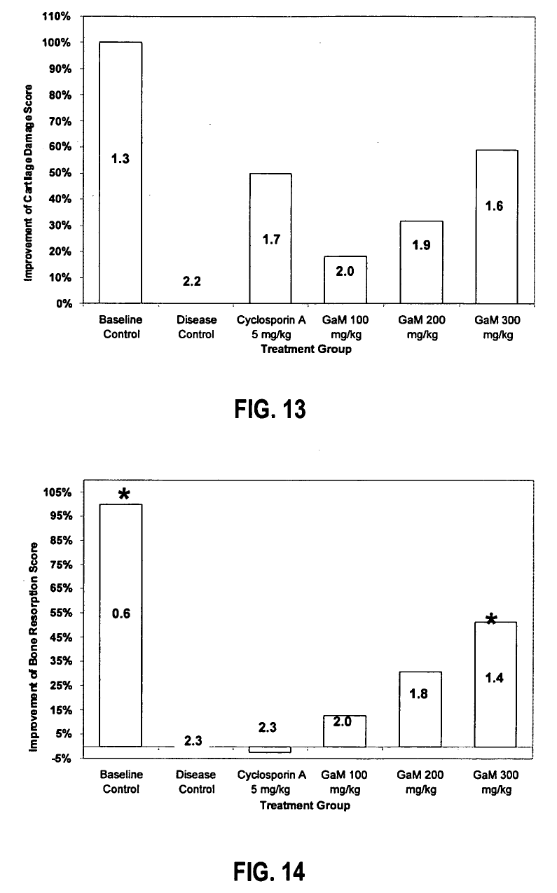

FIG. 13 shows percent improvement of cartilage damage in rats with PGPS-induced chronic arthritis treated with gallium maltolate (GaM), cyclosporin A, or vehicle (normal and disease controls). Results are expressed as the mean percent difference from disease controls

$${\pm SE .}^{•}$$

Results are also expressed numerically as score of cartilage damage on the scale: normal=0, minimal change≦1, mild change≦2, moderate change≦3, marked change≦4, and severe change=5, n=4 rats for baseline control group, n=12 rats for disease control and treatment groups.

FIG. 14 shows percent improvement of bone resorption in rats with PGPS-induced chronic arthritis treated with gallium maltolate (GaM), cyclosporin A, or vehicle (normal and disease controls). Results are expressed as the mean percent difference from disease controls

$${\pm SE .}^{•}$$

Results are also expressed numerically as score of bone resorption on the scale: normal=0, minimal change≦1, mild change≦2, moderate change≦3, marked change≦4, and severe change=5, n=4 rats for baseline control group, n=12 rats for disease control and treatment groups, *p<0.05 compared with disease control group.

In summary, in the chronic model for streptococcal cell wall-induced arthritis, oral gallium delivered as gallium maltolate was safe with no signs of toxicity observed after 35 days of daily administration. Significant dose dependent anti-inflammatory effects on the pannus, cartilage, periosteal proliferation, and bone resorption were observed.

### Serum Gallium Levels for Rheumatoid Arthritis Studies

The following data was compiled from the above described model studies. All sampling was done 1 hour post-dosing.

TABLE 1

| Model | Acute Arthritis | | Chronic Arthritis | | Acute Arthritis | | Acute Arthritis | | Acute Arthritis | |
|---|---|---|---|---|---|---|---|---|---|---|
| Study No. | LATT-1 | | PG-PS/TT-1 | | LATT-2 | | LATT-2 | | LATT-2 | |
| No. of | 4 | | 12 | | 4 | | 4 | | 4 | |
| animals | | | | | | | | | | |
| Dose | 100 | mg/kg | 100 | mg/kg | 100 | mg/kg | 300 | mg/kg | 300 | mg/kg |
| Suspension | Solution | | 1% | MC | 1% | MC | 1% | MC | 1% | MC |
| Duration | 14 | days | 35 | days | 14 | days | 14 | days | 14 | days |
| Pretreatment | 1 | days | 13 | days | 1 | days | 1 | days | 7 | days |
| Fasted prior | No | | Yes | | Yes | | Yes | | Yes | |
| to termination | | | | | | | | | | |
| Fasted prior | No | | Yes | | Yes | | Yes | | Yes | |
| to daily | | | | | | | | | | |
| dosing | | | | | | | | | | |
| Mean | 652 | | 2050 | | 1346 | | 3470 | | 2964 | |
| SD | 210 | | 455 | | 401 | | 704 | | 372 | |
| Serum | | | | | | | | | | |
| Gallium | | | | | | | | | | |
| Concentration | | | | | | | | | | |

1. A method of treating inflammatory arthritis and rheumatic diseases comprising administering to an individual in need thereof, a therapeutically effective amount of gallium, wherein the therapeutically effective amount provides a gallium blood serum level within the range of approximately 50-7000 ng/ml.

2. The method of claim 1, wherein the inflammatory arthritis is selected from rheumatoid arthritis, ankylosing spondylitis, psoriatic arthritis, juvenile rheumatoid arthritis, Reiter's Syndrome and enteropathic arthritis.

3. The method of claim 1, wherein the rheumatic disease is selected from systemic lupus erythematosus, systemic sclerosis and scleroderma, polymyositis, dermatomyositis, temporal arteritis, vasculitis, polyarteritis, Wegener's Granulomatosis and mixed connective tissue disease.

4. The method of claim 1, wherein the gallium is selected from gallium acetate, gallium carbonate, gallium citrate, gallium chloride, gallium fluoride, gallium formate, gallium nitrate, gallium oxylate, gallium oxide and hydrated gallium oxide, gallium phosphate, gallium tartrate, gallium-pyridoxal isonicotinoyl hydrazone, tris(8-quinolinolato)gallium (III), neutral 3:1 gallium complexes of a 3-hydroxy-4-pyrone, gallium (III) complexes of an N-heterocycle, and gallium salt complexes of polyether acids.

5. The method of claim 1, wherein the gallium is administered orally.

6. The method of claim 5, wherein the gallium is a neutral 3:1 gallium complex of a 3-hydroxy-4-pyrone.

7. The method of claim 5, wherein the gallium is a gallium (III) complex of an N-heterocycle.

8. The method of claim 5, wherein the gallium is a gallium salt complex of a polyether acid.

9. A method of preventing pannus formation, comprising administering a therapeutically effective amount of gallium to a patient in need thereof.

10. A method of preventing periosteal proliferation, comprising administering a therapeutically effective amount of gallium to a patient in need thereof.

11. A method of preventing cartilage damage, comprising administering a therapeutically effective amount of gallium to a patient in need thereof.

12. A method of preventing splenomegaly, comprising administering a therapeutically effective amount of gallium to a patient in need thereof.

13. A method of preventing bone resorption due to inflammatory arthritis, comprising administering a therapeutically effective amount of gallium to a patient in need thereof.
